(19) United States
(12) Patent Application Publication
(10) Pub. No.: US 2004/0219511 A1
(43) Pub. Date: Nov. 4, 2004

(54) High-throughput screening assay for chloride channel activity using atomic absorption spectroscopy

(76) Inventor: Dong Cuan Liang, Vancouver (CA)

Correspondence Address: Dong C. Liang, 1001 East Pender Street, Vancouver, BC, V6A 1W2, CA

(21) Appl. No.: 10/833,018

(22) Filed: Apr. 28, 2004

Related U.S. Application Data
(60) Provisional application No. 60/466,688, filed on Apr. 30, 2003.

(51) Int. Cl. C12Q 1/00; G03C 1/005; G03C 1/494

(52) U.S. Cl. 435/4

(57) ABSTRACT

A method for screening of potential modulators of chloride ion channels is described. Flux of chloride is measured indirectly by first precipitating the chloride which has moved out of the cell by addition of an excess of silver ions. Then, the concentration of silver ions left in solution is measured using atomic absorption spectroscopy. This value is then used as a measure of the amount of chloride flux that has occurred.

### RELATED APPLICATIONS

This application claims the benefit of prior filed Provisional Application No. 60/466,688.

### BACKGROUND OF THE INVENTION

An ion channel is a pore formed of one or more protein subunits in the cell membrane. These pores allow the movement of ions in (influx) and out (efflux) of the cell. These channels are generally selective for the movement of a specific ion. Important to the present invention, is the fact that there are ion channels which are selective for the movement of chloride ions. The Cystic Fibrosis Transmembrane Conductance Regulator (CFTR) gene encodes a cAMP activated-chloride channel that is required for proper function of secretory cells in the airway, intestine, pancreas, liver, lungs, and reproductive tract. The CFTR channel is an outward rectifying chloride ion channel. That is, it allows chloride to flow out of the cell. Mutations of such genes are responsible for a variety of diseases, particularly when the mutation results in any loss of channel function.

Channel dysfunction arising from the CFTR gene is most notably associated with the disease Cystic Fibrosis, but it has also been associated with forms of male infertility, polycystic kidney disease, secretory diarrhea, chronic obstructive pulmonary disease, asthma, bronchitis, emphysema, and pneumonia. Thus, chloride channels are a potential target for drug candidates. For example, by using pharmacological intervention to restore normal CFTR channel activity, one can reduce/reverse the effects of a malfunctioning CFTR channel. It has been found that even a 5-10% improvement in chloride conductance is believed to have substantial therapeutic value. Therefore, the need exists for the invention of a high throughput screening (HTS) assay that will effectively and rapidly screen for modulators of chloride channel activity. The present invention was developed using the CFTR channel as a test case, however, the assay could be applied to several other chloride channels.

Current technologies for measuring halide conductance (such as fluorescent indicators) pose problems such as: high background noise, half-life problems, quenching effects, and pH sensitivity. Another technology which shows promise to overcome these problems is the automated patch-clamp, which are now commercially available. However, these systems have definitely not produced as promised in that their throughput is still quite low. Another disadvantage of these systems is the fact that they are only measuring a single cell. It would be more physiologically relevant to measure the activity of a population of cells since cells generally exist in a population inside living organisms. The present invention described here gives an effective HTS method to determine chloride channel activity using atomic absorption spectroscopy. The ion channel activity of a population of cells is measured using a method that overcomes the limitation of the technologies mentioned above. The method and techniques involved will be made clear by way of example using CFTR as the candidate channel.

### SUMMARY OF THE INVENTION

A method is provided to screen for modulators of chloride ion channels using atomic absorption spectroscopy. Flame atomic absorption spectroscopy (FAAS) or graphite furnace atomic absorption spectroscopy (GFAAS) could be used. In one embodiment, cells expressing the chloride ion channel of interest are surrounded with a solution that activates the chloride channels. Varying amounts of potential activators or inhibitors are added to assess their activity. Next, the supernatant is removed from the cells and a known amount of silver ions is added to this solution as silver nitrate. It is important to note that the amount of silver ions that is added is in excess of the chloride ions that are present, the reason for which are described below. Silver ions complex with the chloride ions forming the solid silver chloride. This solid precipitates and can be separated from the liquid phase. Next, the remaining silver ions left in solution are measured using atomic absorption spectroscopy and through well known theory and calculations the amount of chloride that came out of the cells can be determined. Briefly, the chloride present reduces the silver concentration and this reduction can be used to calculate the amount of chloride that came out of the cells. It is important to note that the chloride is not measured directly since measuring chloride ions using atomic absorption spectroscopy is not feasible.

An advantage of this invention is that the experimental methodology described herein provides a way for researchers to accurately determine the therapeutic effects of chloride channel modulating compounds for the purpose of drug discovery. Chloride channels are extremely important to several physiological processes and therefore it is very important to be able regulate or restore the activity of a malfunctioning channel.

### BRIEF DESCRIPTION OF DRAWINGS

Further features and advantages of the invention will be apparent from the following detailed description, given by way of example, of a preferred embodiment taken in conjunction with the accompanying drawing, wherein:

FIG. 1 is a block diagram of the procedure for carrying out the chloride efflux assay.

FIG. 2 is a depiction of the chemical reaction that occurs during the silver chloride precipitation.

### DETAILED DESCRIPTION OF THE INVENTION

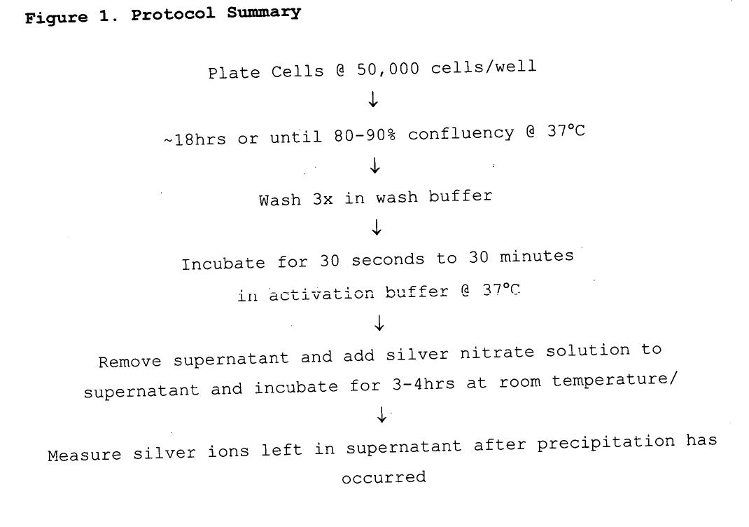

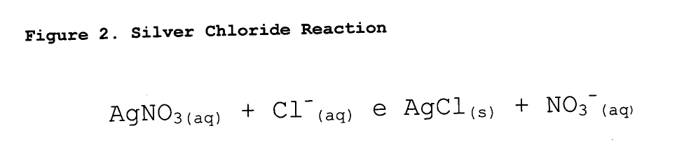

Referring to FIG. 1 and FIG. 2, a description of a preferred embodiment of the invention is shown. Specifics of the invention will be made known by way of example using the CFTR channel as an example.

### TISSUE CULTURE

The cells used for the analysis may be any cell line in which the cells express outwardly rectifying chloride channels, such as the CFTR channel. Common cell lines that may be used include but are not limited to: chinese hamster ovary (CHO) cells, human embryonic kidney (HEK) cells, or fibroblast cell lines. The cells can express the chloride ion channel endogenously or the expressed ion channel can be the result of transfection genes. This assay was developed using a CFTR expressing cell line (T-84); however, its application is not limited to this family of chloride channels.

The cells expressing the ion channel of interest are incubated and cultured by traditional means, all of which are well known to those individuals skilled in the art. For the CFTR assay developed using the T-84 cell line, cells were grown in 1:1 Dulbecco's modified eagle's media (DMEM) and Ham's F-12 medium, supplemented with 10% fetal calf serum (FCS) at 37° C. celsius , in 5% CO₂. A digestive enzyme, such as trypsin, is used to break the protein bonds between the cells and the culture vessels. The cells are removed from the culture vessels and were plated at a density of approximately 50,000 cells/well in 96-well microplates and incubated at 37° C., 5% CO₂ until 80-90% confluency is attained. The 96-well plates typically have some type of special surface treatment which allows for proper cellular adhesion. The cells are allowed to incubate at 37° C. for a minimum 12-hour period (typically 18 hours). The exact experimental incubation period will depend on the desired final cell density, the type of cell line used, and on the level of ion channel expression. The purpose of this incubation period is to allow the cells to grow, express the ion channels, multiply to increase the cell density in the microplate, and to allow cells to adhere to the surface of the microplate wells.

### ASSAY

The cell monolayer on the bottom of each well is then washed three times with a wash buffer. The wash buffer does not contain any chloride ions. It is an isotonic solution which serves to remove any extra-cellular chloride ions. These types of steps can be done using either an auto-sampler or it can be done manually, allowing the injection and subsequent aspiration of buffer solution into each sample well. This buffer also contains a nutritional supplement such as glucose to help feed the cells. The chemicals and biological substances used in this buffer are all commercially available and familiar to persons skilled in the art. Other cell lines may require other ingredients and/or additional salts to create the best condition for the health of the cells.

Channel activation and testing of compounds for activity on the ion channels occurs next. At this point the activation buffer is added to the cell monolayer. This buffer is the same as the wash buffer except it contains the following additions:

(a) A known channel activator (in the case of the t-84 cell line the agonist forskolin was used).

(b) the test compound in varying concentrations, being the candidate compound of interest which may serve to further activate the channel, or inhibit channel activity.

An agonist is a specific compound that acts by binding to the receptor site of the ion channel causing a reaction that mimics a natural chemical messenger or a membrane charge stimulus. The effect of the agonist forskolin on the CFTR channel is activation of the channel leading to chloride efflux. This type of up regulation of channel activity generates a window of detection, such that if you added a compound which blocked CFTR activity that you would see a reduction in chloride efflux. This application can also be manipulated to detect channel activators. For example, the activity of a very weak agonist drug may be elucidated by performing this assay at increasing concentrations of a test compound in the presence of a low fixed concentration of Forskolin. Therefore, this drug discovery application may be used to screen for chloride channel agonists, antagonists, and neutral candidate compounds which have no appreciable effect.

The control samples include, but are not limited to, the following:

(a) a negative control indicating the basal chloride ion flux in the absence of any known agonist or test compound; and

(b) a positive control indicating the chloride ion flux in a medium containing a known agonist, but in the absence of any test compound.

To determine the activity of a compound the prepared unknown and control samples (in activation buffer) are added to the cell monolayer. This incubation period may vary experimentally, from seconds to several minutes, depending on the cell line. After this incubation period, the cells are then isolated from the extracellular solution. Using the T84 cell line expressing CFTR, the following steps were taken to complete the assay. These steps may need to be modified slightly for other cell lines. To 200 μL of the extracellular solution, 30 μL of a silver solution (50 ppm silver as silver nitrate) was added. As per FIG. 2, the silver ions present react with the chloride ions to form the solid silver chloride. This precipitate was allowed to settle for 3-4 hours. The free silver ions in this solution were then analyzed using atomic absorption spectroscopy (best results were achieved using the ICR series from Aurora Biomed, Vancouver). Using the ICR 8000 or the ICR 12000 coupled with automated liquid handling techniques allows the assay to be done in a high throughput format.

Halide ions, including chloride, are known to be a highly reactive ion species. Referring to FIG. 2, the chemical reaction between chloride and silver immediately produces a stable silver chloride solid that precipitates out of solution. This theory is well known and has been studied extensively. With an understanding of this theory one can calculate the concentration of chloride that was in the supernatant after the activation period, which would have been due to the chloride channel activity. This calculation is relatively simple for one skilled in the arts and takes into account such things as the solubility constant of silver chloride, the amount of silver ions added, and the exact volumes involved. The reader is encouraged to consult basic chemistry texts which cover such topics as equilibrium, solubility, and thermodynamics.

### AAS

Atomic absorption spectroscopy (AAS) is a well-known technique for elemental chemical analysis. Flame atomic absorption spectroscopy (FAAS) uses a flame furnace to first vaporize the solute ions and then measure the concentration of gas-phase atoms using the absorption of light. The detection level of silver using the ICR 8000 (Aurora Biomed) is very low, with a dynamic range of 0.02 ppm to 4 ppm. Such automated instrumentation increases the throughput of assays by using microsyringe autosampling. A graphite furnace atomic absorption spectroscopy (GFAAS) operates on a similar premise but has even greater sensitivity than FAAS. However, GFAAS is only appropriate with extremely low volumes of sample. Either method can be applied to accurately measure chloride flux activity through the ion channel using the silver chloride precipitation method described above.

The concentration of silver ions remaining in solution is thereby measured with an atomic absorption spectrometer. Therefore, we claim an invention that is able to determine the activity of the chloride channel.

### DATA PROCESSING

The method described here can be used to determine whether a candidate compound, that is designed specifically to target chloride channels, is an antagonist (channel blocker), an agonist (channel activator), or has no effect on its activity (neutral). For example, if the addition of the test compound results in a lower concentration of chloride ions than the basal flux, then this would indicate that the compound is an activator of chloride channels, by increasing the efflux of ions. Alternatively, if the test compound results in a higher concentration of chloride ions in the cell than found basally, it indicates that the compound is a blocker of the chloride channel, by decreasing the efflux. If the addition of a test compound results in no more or no less chloride ions than in the sample without the addition of the compound, then this would indicate that the compound is a non-blocker and non-activator of the chloride channel, or neutral in effect on the ion flux. Furthermore, this application is useful in drug safety screening to determine whether drugs for other targets may also have unwanted or adverse effects on chloride channel activity.

What I claim as my invention is:

1. A method for identifying compounds that potentially modulate the activity of a chloride ion channel, comprising:
a. washing cells expressing said ion channel in a first isotonic solution that does not contain any chloride ions;
b. incubating said cells in a second isotonic medium that does not contain any chloride ions so as to allow chloride to move out of the said cells via said ion channel;
c. separating the extracellular solution from said cells and adding silver ions to said extracellular solution so as to produce the solid precipitate silver chloride; and
d. measuring silver ions in the said extracellular solution which did not form silver chloride using one of atomic absorption spectrometer and graphite furnace atomic absorption spectrometer.

2. The screening method according to claim 1, wherein the said second isotonic medium contains a known channel activator.

3. The screening method according to claim 1, wherein the said second isotonic medium contains a test compound which is a potential modulator of ion channel activity.

4. The screening method according to claim 1, wherein the said second isotonic medium contains a channel agonist.

5. The screening method according to claim 1, wherein said cells are selected from groups of cells derived from a human or an animal.

6. The screening method according to claim 1, wherein said cells are selected from the group consisting of chinese hamster ovary cells, human embryonic kidney cells, T84 cells, Calu-3 or fibroblast cells, and cardiac cells or epithelial cells.

7. The method of claim 1, wherein said cells express the chloride ion channel endogenously or as a result of transfection.

8. The method of claim 1, wherein said chloride ion channel is the product of the cystic fibrosis transmembrane conductance regulator gene.

9. The screening method according to claim 1, wherein said cells are placed into one or more wells of a multi-well microplate.

10. A screening method according to claim 4, wherein the microplate comprises 96, 384, or 1536 wells.

11. A screening method according to claim 5, wherein the microplate is compatible with automated sample handling apparatus and analysis apparatus.

12. A screening method according to claim 6, wherein the apparatuses are computer-controlled.

13. A screening method according to claim 1, further comprising, adding a known chloride channel activator simultaneously with addition of a test compound in the second isotonic medium, wherein said test compound is being screened for its ability to inhibit or activate the said chloride channel.

14. A screening method according to claim 3, wherein the test compound activates the chloride channel.

15. A screening method according to claim 3, wherein the test compound antagonizes the activity of the chloride channel.

16. A screening method according to claim 3, wherein the test compound potentiates the activity of the chloride channel.

17. The screening method according to claim 1, wherein the said silver ions added exceed the chloride ions present.

18. The screening method according to claim 1, wherein the said silver ions are added as a solution of silver nitrate.

19. A method for monitoring chloride movement out of a cell comprising:
a. removing all chloride ions from a solution of said cells;
b. adding a test compound to said cells which may inhibit or increase chloride efflux;
c. separating extracellular solution from said cells;
d. adding an excess of silver ions relative to the chloride ions to the said extracellular solution;
e. measuring the remaining free silver ions in the said extracellular solution; and
f. calculating the amount of chloride that crossed the said cell membranes.
